(19) United States
(12) Patent Application Publication
(10) Pub. No.: US 2026/0159904 A1
(43) Pub. Date: Jun. 11, 2026

(54) RAPID METHOD TO DETERMINE EFFECTIVE ORAL ANTI-BACTERIALS

(71) Applicants: Guy Adami, Brookfield, IL (US); Stefan Joshua Green, Chicago, IL (US)

(72) Inventors: Guy Adami, Brookfield, IL (US); Stefan Joshua Green, Chicago, IL (US)

(21) Appl. No.: 19/395,207

(22) Filed: Nov. 20, 2025

Related U.S. Application Data
(60) Provisional application No. 63/722,861, filed on Nov. 20, 2024.

(51) Int. Cl. C12Q 1/689

(52) U.S. Cl. CPC C12Q 1/689; C12Q 2600/136

(57) ABSTRACT

Methods for rapidly testing agents for activity against oral microorganisms using naturally shed oral biofilm obtained directly from a subject. Samples of the shed oral biofilm are incubated with and without a test agent, preferably without subculturing and for a short incubation period (e.g., 4 hours or less). Following incubation, levels of one or more nucleic acid markers, such as ribosomal RNA, messenger RNA, or ribosomal DNA, are determined. A decrease in the level of the nucleic acid marker in the agent-treated sample relative to the control indicates antimicrobial activity of the agent. The methods provide a rapid and reproducible ex vivo assay capable of identifying antimicrobial agents that target microorganisms within the oral microbial community.

### CROSS-REFERENCE TO RELATED APPLICATIONS

Priority is hereby claimed to provisional application Ser. No. 63/722,861, filed Nov. 20, 2024, which is incorporated herein by reference.

### SEQUENCE LISTING

The Instant Application contains a Sequence Listing which has been submitted electronically in XML format and is hereby incorporated by reference in its entirety. The XML copy, created on Nov. 14, 2025, is named “SEQ_LIST-110695002.xml” and is 2,822 bytes in size.

### BACKGROUND

Oral diseases, including dental caries and periodontal disease, are seemingly non-life-threatening diseases that are economically costly and reduce quality of life for millions of people in the United States, especially among the elderly. Oral diseases and/or disorders can significantly impact a person's overall health. Research has shown that oral bacteria may contribute to increased risk of systemic disease and conditions, from cardiovascular disease to preterm birth.

Oral biofilms such as oral plaque play important causative roles in the development of caries and periodontal diseases. Oral plaque is a complex and diverse microbial consortium consisting of hundreds of bacterial species embedded in polymer matrix and adhered tightly to tooth or oral mucosal surfaces. Changes in the microenvironment can drive alterations in the composition and metabolic activity of the oral biofilm at a site, resulting in a local microbiome that can be deleterious to the host. There is also ample evidence that less abundant pathogenic oral taxa can have large effects on host response such as inflammation, and thus promote dysbiotic states. Moreover, a large number of taxa in the appropriate combinations may be dysbiotic and promote disease. The net result is that induced change in levels of multiple taxa can result in disease at a site. One part of maintaining health is inhibiting these changes or reversing them.

Compared with their planktonic counterparts, the biofilm cells possess elevated virulence and are highly resistant to antimicrobial agents. While mechanical plaque elimination with assorted devices remains the primary and most widely accepted means to maintain good oral health and to reduce plaque-mediated diseases, other options are used. Chemotherapeutic agents with a variety of direct antimicrobial mechanisms have been beneficial. However, long-term use of broad-spectrum antibiotics can suppress resident oral microflora and permit overgrowth of pathogenic or opportunistic organisms, and anti-bacterials like chlorhexidine can act as irritants. Agents that change the microenvironment can drive alterations in the composition and metabolic activity of the oral microflora resulting in one that may either be deleterious or be beneficial to the oral cavity of the host. Therefore, disease can be prevented not only by targeting the putative pathogens directly by antimicrobial strategies, but also by interfering with the selection pressures responsible for their enrichment. There is a need for agents that promote health associated bacterial populations in oral biofilm.

Plant-derived compounds offer an abundant source of antimicrobial agents that find wide acceptance by the public as “natural products.” In recent years, “phytomedicines” have gained significant popularity in the U.S. and various natural extracts have been incorporated into oral hygiene or therapeutic products. In addition to their demonstrated in vitro antimicrobial activity, many demonstrated potential in clinical utility. The American cranberry (Vaccinium macrocarpum) is a rich source of polyphenolic bioactives, particularly the proanthocyanidins (PACs), which may contribute to human health. This natural product has been popular in the self-treatment of urinary tract infections and has the potential to aid in control of oral biofilm. The PACs in cranberries have been shown to possess antimicrobial and anti-adhesion effects. In vitro studies have shown that cranberry extract and its PACs interfere with cariogenic Streptococcus mutans biofilms by inhibiting glucosyltransferases and adherent glucan synthesis, thereby disrupting bacterial biofilms. Cranberry components have also been found to inhibit periodontal pathogens in vitro. Wu et al. (“Beverages containing plant-derived polyphenols inhibit growth and biofilm formation of Streptococcus mutans and children's supragingival plaque bacteria.” Beverages 2021, 7, 43) have reported that US-marketed cranberry juice drinks (CJC, containing 27% juice) caused rapid cellular aggregation and inhibited the ex vivo growth and biofilm formation of oral bacteria. When children's plaque samples were cultured ex vivo and exposed to CJC, loosely attached biofilms were noted and were easily rinsed off surfaces. This showed that CJC's inhibition extended from affecting single species bacteria to the complex plaque biofilm community. Pre-clinical testing of natural products such as those containing cranberry juice is an attempt to predict clinical efficacy.

Numerous models have been developed to assess the efficacy of natural antimicrobial formulations. The majority of bioactivity studies have been conducted in vitro against artificial oral biofilms made of a single or several species of bacteria. However, these models do not replicate the complexity of the human oral biofilm community, which consists of hundreds of bacterial species in the oral cavity, making results difficult to interpret.

Efforts to mimic the oral environment have included the establishment of stable oral bacterial microcosms representative of the multitude of bacteria at relevant sites, such as subgingival and supragingival sites of the oral cavity; coating of artificial surfaces, such as hydroxyapatite or dental materials, with pooled cell free human saliva; utilization of dynamic continuous growth systems with continual replenishment of culture media; and many others. (See e.g., van de Sande et al. “An in vitro biofilm model for enamel demineralization and antimicrobial dose-response studies.” Biofouling 2011, 27, 1057-1063; Fernández et al. “The effect of inoculum source and fluid shear force on the development of in vitro oral multispecies biofilms.” J Appl Microbiol 2017, 122, 796-808; Baraniya et al. “Modeling Normal and Dysbiotic Subgingival Microbiomes: Effect of Nutrients.” J Dent Res 2020, 99, 695-702.) Research has shown that with the appropriate growth medium and the addition of human serum or specific carbohydrates, one can establish oral biofilm consortium that preserves most of the natural microbial taxa present at oral sites (Edlund et al. “Uncovering complex microbiome activities via metatranscriptomics during 24 hours of oral biofilm assembly and maturation.” Microbiome 2018, 6, 217; Jiang et al. “Manipulation of Saliva-Derived Microcosm Biofilms To Resemble Dysbiotic Subgingival Microbiota.” Appl Environ Microbiol 2021, 87, e02371-20; Baraniya et al. “Optimization of conditions for in vitro modeling of subgingival normobiosis and dysbiosis.” Front Microbiol 2022, 13, 1031029). Formation of these biofilms seemed to mimic similar steps that occur with subgingival biofilm formation in vivo. Using subgingival plaque samples pooled from healthy sites or periodontal disease sites, subsequent cultures retained the majority of taxa from the original sample sites over several days. These artificially formed biofilms can be sourced from oral mucosa surfaces, dental subgingival plaque, or saliva (see e.g., Baraniya et al. “Modeling Normal and Dysbiotic Subgingival Microbiomes: Effect of Nutrients.” J Dent Res 2020, 99, 695-702; Baraniya et al. “Optimization of conditions for in vitro modeling of subgingival normobiosis and dysbiosis.” Front Microbiol 2022, 13, 1031029; Cieplik et al. “Microcosm biofilms cultured from different oral niches in periodontitis patients.” J Oral Microbiol 2019, 11, 1551596; Edlund et al. “An in vitro biofilm model system maintaining a highly reproducible species and metabolic diversity approaching that of the human oral microbiome.” Microbiome 2013, 1, 25; Ziemyte et al. “Personalized antibiotic selection in periodontal treatment improves clinical and microbiological outputs.” Front Cell Infect Microbiol 2023, 18, 1307380; Parga et al. “The quorum quenching enzyme Aii20J modifies in vitro periodontal biofilm formation.” Front Cell Infect Microbiol 2023, 13, 1118630; Lamont et al. “Modified SHI medium supports growth of a disease-state subgingival polymicrobial community in vitro.” Mol Oral Microbiol 2021, 36, 37-49). These oral microcosms are difficult to establish and can change with time but are generally considered to better mimic the in vivo environment compared to single-species biofilms. Freezing of donor oral microbial plaque samples has been used to facilitate the process, but it may affect the subsequent biofilm formation.

There is a need to develop superior models that can efficiently mimic the in vivo oral cavity microenvironment to characterize mechanisms and mode of actions of antimicrobial agents against oral microbial biofilms. The development of such models may aid in selection and design of clinical oral anti-bacterial trials.

### SUMMARY

The present disclosure provides methods for testing agents for activity to modulate oral microorganisms using shed oral biofilm obtained directly from a subject. In certain aspects, the method comprises:
(a) providing a sample from a subject comprising shed oral biofilm;
(b) incubating a first portion of the sample with an agent, and a second portion of the sample without the agent, under a condition to yield a first incubated sample and a second incubated sample, and preserving a baseline portion of the sample prior to incubation; and
(c) determining level of at least one biological marker in the first incubated sample, the second incubated sample, and the baseline portion.

In some embodiments, the shed oral biofilm sample is obtained in the form of saliva, and the sample may be washed prior to incubation to enhance reproducibility. In certain embodiments, the incubation is carried out in a rich medium capable of supporting microbial metabolic activity.

In preferred embodiments, the incubation is carried out without subculturing the microorganisms. The incubation may be conducted for a short period of time, such as four hours or less, or even one hour or less. The incubation may be conducted under anaerobic or oxygen-limited conditions.

Following incubation, level of at least one biological marker is determined in each of the incubated samples and the baseline portion. The biological marker may include, but is not limited to, a nucleic acid, a protein, a metabolite, or a combination thereof.

The nucleic acid marker may include ribosomal RNA (rRNA), messenger RNA (mRNA), ribosomal DNA (rDNA), another DNA-based marker, or a combination thereof.

In certain embodiments, the nucleic acid marker comprises full-length bacterial 16S rRNA or a region thereof (e.g., V1-V2, V3, or V4 regions). In certain embodiments, the nucleic acid marker comprises one or more bacterial mRNAs indicative of gene expression changes in response to the agent. In certain embodiments, the nucleic acid marker comprises full length bacterial 16S rDNA or a region thereof.

Determining the level of the nucleic acid marker may comprise isolating total RNA or total DNA from the incubated sample.

The level of the nucleic acid marker may be determined by any suitable nucleic-acid-based method, including, but not limited to, reverse-transcription PCR, quantitative PCR, digital PCR, next-generation sequencing, and RNA sequencing.

In certain embodiments, a change in the level of the biological marker in the first incubated sample relative to the second incubated sample or to the baseline portion indicates that the agent modulates one or more oral microbes, including inhibiting, reducing, or increasing one or more oral microbial taxa. For example, a decrease in the level of the at least one nucleic acid marker in the first incubated sample compared to the second incubated sample or the baseline portion indicates activity to inhibit oral microbes. Such inhibition may include suppression of microbial activities as reflected by reduced levels of mRNA markers.

The disclosed methods provide a rapid and reproducible ex vivo assay for identifying agents that modulate oral microbes, including agents that selectively inhibit or stimulate one or more taxa within the oral microbial community.

The objects and advantages of the disclosure will appear more fully from the following detailed description of the preferred embodiment of the disclosure made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E. Differences in 16S rRNA levels in ex vivo samples from three different subjects identified as C_, G_ and L_. Each point represents one ex vivo sample incubated for 4 hours in test media without an anti-bacterial treatment, performed in duplicate. (FIG. 1A) Chao1 index, G_ vs L_ p<0.00014, C_ vs L_ p<0.00025. (FIG. 1B) Shannon index, G vs L_ p<0.14, C_ vs L_ p<0.0054. (FIG. 1C) Beta Diversity Bray Curtis distances, PERMANOVA G_ vs L_ F-value 29.8916, R-squared=0.74932, p<0.002. PERMANOVA C_ vs L_ F-value 36.9, R-squared=0.787, p<0.006. G_ vs C_, F-value 4.24, R-squared=0.29, p<0.01. (FIG. 1D) Bar plot indicating relative level of 16S rRNA transcripts from the various taxa from different subjects' ex vivo samples. (FIG. 1E) Heat map of nonsupervised clustering of samples based on taxa 16S rRNA levels. Samples clustered based on taxonomic profiles.

FIG. 2. The effect of different agents on the abundance of bacterial 16S rRNA transcripts in shed biofilm ex vivo samples. (Panel A) Chao1 index showed no statistically significant differences. (Panel B) Shannon index: vancomycin (Van) vs control (Con) p<0.0000014, penicillin G (Pen) vs. control (Con), p<0.000075, cranberry (Cra) vs control (Con), p<0.32. (Panel C) Beta diversity based on Bray Curtis distances, 3 agents and the control. (Panel D) Beta diversity: 10 μg/mL vancomycin exposure vs control, PERMANOVA F-value 11.5543, R-squared=0.29969, p<0.001. (Panel E) Beta diversity: 5 μg/mL penicillin G exposure vs control, PERMANOVA F-value 11.5543, R-squared=0.29969, p<0.001. (Panel F) Beta diversity based on Bray Curtis distances, 3 agents and the control (Con), Beta diversity 17% cranberry juice exposure vs control, PERMANOVA F-value 2.42, R-squared=0.082, p<0.010. (* indicates p<0.05).

FIGS. 3A-3C. Differential abundance of 16S rRNA after incubation of shed biofilms from three subjects tested individually. (FIG. 3A) After exposure to vancomycin, 16S rRNA assigned to these species were identified by DeSeq2 as differentially abundant compared to the control (FDR<0.10). (FIG. 3B) After exposure to penicillin G, 16S rRNA assigned to these species were identified by DeSeq2 as differentially abundant compared to the control (FDR<0.10). (FIG. 3C) After exposure to 17% cranberry juice, 16S rRNA assigned to these species were identified by DeSeq2 as differentially abundant compared to the control (FDR<0.10). The Y-axis represents the percentage of total reads assigned to each taxon for the differentially abundant 16S rRNA transcripts. All subjects' samples were collected and tested on three separate days. The control contained 0.4% DMSO vehicle, consistent with the antibacterial treatment conditions.

FIG. 4. Differential abundance of 16S rRNA transcripts and 16S rDNA (the 16S rRNA gene) after 4-hour incubation of shed biofilms with the 3 agents (Van, Pen, and Car) or vehicle (Con). Total 16S rDNA for each incubation was normalized to the relative level of that molecule in the vehicle-treated control. Total 16S rRNA was normalized to the total 16S rRNA in the control. “*” indicates differential abundance versus control based on Student t-test (p<0.05).

FIG. 5. Differential abundance of bulk DNA and RNA after 4 hours incubation of shed biofilms with the 3 agents (VAN, PEN, and CRA). N=18 for all two-way RNA comparisons with the exception of PEN versus controls which included 16 samples. DNA pairwise comparisons had N=12. “*” indicates differential abundance of test samples versus baseline (BASE), preserved at assay start. “**” represents differential abundance versus negative control 0.4% DMSO vehicle treated samples incubated for 4 hours (CON). Both comparisons were based on Mann Witney test (p<0.05). (Left panel) RNA: CON vs. BASE, p<0.00174; VAN vs. BASE, p<0.0124; PEN vs BASE, p<0.267; CRA vs. BASE, p<0.0028; VAN vs. CON, p<0.064; PEN vs. CON, p<0.00148; CRA vs. CON, p<0.00200. (Right panel) DNA: CON vs. BASE, p<0.00193; VAN vs. BASE, p<0.073; PEN vs. BASE, p<0.186; CRA vs. BASE, p<0.024; VAN vs. CON, p<0.400; PEN vs. CON, p<0.00496; CRA vs. CON, p<0.0028.

FIG. 6. Differential abundance of 16S rRNA after 1-hour incubation of shed oral biofilms from five saliva donors tested individually. After 1-hour exposure to 1 mM myricetin, 16S rRNA assigned to the four taxa identified as differentially abundant in the earlier 4-hour assay (see Table 6) was evaluated using the same 16S rRNA amplicon analysis. Based on a paired Student's t-test, two of the four taxa (indicated by “*”) were differentially abundant compared to the control (p<0.05). The control samples contained 0.4% DMSO vehicle, as did the antibacterial tests.

### DETAILED DESCRIPTION

### Definitions

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite articles “a” and “an” mean “one or more” unless explicitly stated otherwise.

As used herein, the term “or” is an inclusive “or” operator and is equivalent to the term “and/or” unless the context clearly dictates otherwise.

All combinations of method or process steps as used herein can be performed in any order, unless otherwise specified or clearly implied to the contrary by the context in which the referenced combination is made.

The methods disclosed herein can comprise, consist of, or consist essentially of the essential elements and limitations of the method described, as well as any additional or optional ingredients, components, or limitations described herein or otherwise useful in microbiology, biochemistry, and/or mycology. The disclosure provided herein may be practiced in the absence of any element or step which is not specifically disclosed herein.

The term “Nucleic acids” as used herein refers to biopolymers comprising chains of monomeric nucleotides, which serve as the fundamental molecules for the storage, transfer, and expression of genetic information. These molecules include deoxyribonucleic acid (DNA) and ribonucleic acid (RNA) and may encompass naturally occurring forms as well as synthetic or modified analogs. Nucleic acids can exist in single-stranded or double-stranded configurations and may include coding and non-coding sequences. Additionally, the term encompasses fragments, variants, and derivatives of nucleic acids, such as complementary DNA (cDNA), messenger RNA (mRNA), ribosomal RNA (rRNA), transfer RNA (tRNA), synthetic oligonucleotides, and chemically modified nucleotides. Nucleic acids may also include sequences labeled with chemical or biological markers for use in detection, quantification, or functional analysis.

As used herein, “shed oral biofilm” refers to any naturally detached or dislodged microbial aggregate originating from oral biofilms present on any oral/oral pharyngeal surface, including but not limited to teeth, gingiva, tongue, palate, tonsils, throat, and oral mucosa. The term includes multicellular aggregates, microcolonies, or fragments of oral plaque or mucosal biofilm that become suspended in saliva or other oral fluids. The term is not limited to saliva and encompasses shed biofilm present in rinsates, swabs, expectorated material, or any collected oral specimen.

As used herein, “without subculturing” means that the microorganisms in the shed oral biofilm sample are not grown, expanded, or propagated on culture media prior to testing. Incubation is performed directly on the naturally collected microbial aggregates.

As used herein, “modulating” refers to altering the level, activity, viability, growth, gene expression, or relative abundance of one or more oral microorganisms or microbial taxa. Modulating includes inhibiting, reducing, increasing, or stimulating the growth or activity of such microbial taxa.

### Method to Determine Effective Oral Antimicrobial Agents

Periodontal disease and tooth decay are generally non-life-threatening diseases, yet they are economically costly and reduce quality of life in millions of individuals in the US, particularly among the elderly. These diseases are largely driven by specific bacterial species that inhabit the oral cavity. Currently, no antibacterial agents are available that selectively target the microorganisms responsible for these conditions. Instead, broad-spectrum anti-bacterials in the form of mouth rinses, such as those containing chlorohexidine, are used to target harmful bacteria such as Tannerella forsythia and others. However, such treatments also eliminate beneficial oral bacteria, including those involved in nitrate reduction pathways that generate metabolites leading to nitric oxide production, which contributes to blood pressure regulation. Thus, there remains an unmet need for antibacterial agents that selectively inhibit disease-associated oral bacteria.

Most current research on oral antibacterial agents relies on studies in liquid culture or with artificial plaque or biofilm systems. In these approaches, individual bacterial strains or defined bacterial consortia are isolated from human samples, cultured under laboratory conditions, and then treated with test agents to assess cell viability. However, the results of these assays often fail to predict outcomes in the natural oral environment, as the bacterial populations in vivo exist in complex, multispecies biofilms. Consequently, agents identified in simplified culture systems frequently do not exhibit the expected specificity or efficacy when evaluated in human studies.

Disclosed herein is a screening method for identifying agents that modulate oral microorganisms (e.g., antimicrobials) using mixtures of oral microorganisms present as an Oral Bacterial Consortium (OBC). The OBC is used immediately after collection, without any subculturing. The oral microorganisms may be collected in the form of shed oral biofilm, which comprises naturally detached or dislodged oral biofilm aggregates originating from any oral surface.

In some embodiments, the sample comprising shed oral biofilm is obtained in the form of saliva. However, the disclosure is not limited to saliva, and the shed oral biofilm sample may be obtained from any oral specimen containing microbial aggregates. In certain embodiments, the sample may be obtained from a mouth rinse or oral wash collected from the subject, provided that the sample contains shed oral biofilm aggregates. In certain embodiments, saliva-derived aggregates are used as surrogates to test dental plaque or oral biofilm, in general, susceptibility or response to antibacterial or probiotic agents. Using shed oral biofilm from saliva can improve reproducibility among different sample collectors. Salivary microbial aggregates largely consist of fragments of oral plaque or similar structures dislodged from tooth surfaces, gingiva, tongue, or mucosal epithelia, and remain in saliva only transiently before being swallowed.

After collection, the shed oral biofilm samples are exposed immediately to one or more test agents without subculturing. Preferably, incubation is conducted under anaerobic conditions. In some embodiments the incubation may be performed under substantially oxygen-free conditions. Preferably, the incubation period is 4 hours or less (e.g., about 1 to 4 hours, or less than 1 hour).

To enhance reproducibility, the method may include washing the shed oral biofilm aggregates before incubation. Pooling of samples from multiple donors may be avoided.

In some embodiments, the incubation is carried out in a rich medium. The rich medium may be any nutrient-containing medium that supports metabolic activity, transcription, or survival of microorganisms in the shed oral biofilm sample. Exemplary rich media include, but are not limited to, SHI medium, Brain Heart Infusion (BHI), Tryptic Soy Broth (TSB), and modifications thereof. In certain embodiments, SHI medium is used. In certain embodiments, sheep blood is omitted from the medium, and serum components such as fetal bovine serum may be replaced with bovine serum albumin (BSA).

Following incubation, the reaction is terminated, and one or more biological markers in the treated samples are quantified. The biological marker may include, but is not limited to nucleic acids, proteins, metabolites, or combinations thereof.

The nucleic acid marker may include, but is not limited to, full-length or specific regions of ribosomal RNA (rRNA), messenger RNA (mRNA), ribosomal DNA (rDNA), or other DNA-based markers. Determining the level of a nucleic acid marker may include isolating total RNA or total DNA from the incubated sample. The method may further include amplifying selected nucleic acid regions using sequence-specific primers and analyzing the resulting amplicons by next-generation sequencing (NGS). In other embodiments, the level of a nucleic acid marker may be determined using quantitative PCR (qPCR), reverse-transcription qPCR (RT-qPCR), digital droplet PCR (ddPCR), isothermal amplification techniques (e.g., LAMP, RPA), or hybridization-based assays such as Northern blotting, Southern blotting, microarrays, or related technologies. Additional suitable approaches include capillary electrophoresis, RNA sequencing (RNA-Seq), whole-genome or metagenomic sequencing, amplicon sequencing, direct RNA sequencing, single-molecule sequencing, or any amplification-free or detection-based nucleic acid quantification method known in the art or developed in the future.

The test agent may reduce or increase the abundance of the total microbial population, a subset of the oral microbial community, or one or more specific taxa, as determined by changes in nucleic acid marker levels.

In one embodiment, the method comprises assaying bacterial 16S rRNA to generate a profile of the oral bacterial community, including its composition, diversity, and relative taxonomic abundance. The assay may target full-length 16S rRNA molecules or specific regions, including, but not limited to V1-V2, V3, and V4 regions.

In another embodiment, the method comprises assaying mRNA to assess changes in gene expression in response to the test agent.

Quantification of RNA may be performed using any suitable molecular technique known in the art or later developed. For example, RNA may be reverse-transcribed into complementary DNA (cDNA) and amplified using primers targeting full-length 16S rRNA or conserved or variable regions thereof, followed by NGS. Alternatively, RNA sequencing (RNA-Seq) may be used to quantify both 16S rRNA and mRNA transcripts.

In another embodiment, the method comprises assaying bacterial 16S rDNA using NGS or any other suitable method known in the art or developed in the future.

The nucleic-acid-based assay described herein may also be applied to characterize bacterial or fungal gene expressions in disease states, such as oral cancer. For example, saliva may be collected, incubated for 1-2 hours, and processed for mRNA extraction.

Contrary to earlier assumptions that rRNA and rDNA would not undergo rapid turnover, the present disclosure demonstrates that incubation of shed oral biofilm with agents such as penicillin G or 17% cranberry extract can lead to loss of nucleic acid material, including DNA, relative to baseline samples with no incubation.

A distinctive feature of this method is that RNA levels are measured directly in the intact shed-plaque aggregates after exposure to the test agents, without any subculturing. A decrease in ribosomal RNA occurring within less than 4 hours is used as an indication of antibacterial activity. It was previously believed that such measurement would not be reproducible without subculturing, even within samples from the same individual. The method disclosed herein is specifically designed to minimize that variability. It is further shown that decreases in ribosomal RNA can be detected within a 4-hour period, despite the fact that ribosomal RNA has historically been considered highly stable, even in nonviable bacteria, based on studies in model organisms such as E. coli (Masters et al. “Effect of stress treatments on the detection of Listeria monocytogenes and enterotoxigenic Escherichia coli by the polymerase chain reaction.” J. Appl. Bacteriol. 1994, 77, 73-79).

The agents that can be tested by the disclosed method are not limited, and any compound or composition suspected of affecting the viability, activity, growth, metabolism, transcriptional state, or nucleic acid abundance of oral microorganisms may be evaluated. Examples of suitable agents include, but are not limited to, antibiotics (e.g., β-lactams, glycopeptides, macrolides, tetracyclines, fluoroquinolones, metronidazole, or combinations thereof), antimicrobial peptides, antiseptics (e.g., chlorhexidine, cetylpyridinium chloride, essential oils), natural products or botanical extracts (e.g., cranberry juice or extract, polyphenols, flavonoids, plant-derived antimicrobials), probiotics, prebiotics, postbiotics, synbiotics, bacteriocins, enzymes, quorum-sensing inhibitors, biofilm-disrupting agents, small-molecule inhibitors, metal ions, nanoparticle-based antimicrobials, and formulations thereof. In certain embodiments, the agent may selectively inhibit one or more disease-associated oral taxa, such as periodontal pathogens or cariogenic bacteria. In other embodiments, the method may be used to identify compounds that preserve or promote beneficial oral taxa, modulate community composition, or shift a dysbiotic oral microbiome toward a more health-associated state.

As an example, and as described in detail in the Example section, the disclosed assay was used to identify bacterial targets of cranberry juice, a natural product with known antibacterial properties in the gastrointestinal tract. Diluted cranberry juice was tested for its ability to suppress RNA transcription in freshly isolated oral bacterial aggregates. Cranberry juice reproducibly reduced rRNA levels in a range of bacterial species within individual donors and across a group of four independent donors, indicating shared target taxa. The positive controls, penicillin G and vancomycin, selectively inhibited Gram-positive bacteria consistent with known in vivo activity. Inhibition is defined as blocking accumulation or decreasing quantities of RNA or DNA specific to that taxa. Together, these findings demonstrate that the disclosed rapid ex vivo assay provides an effective tool for screening antibacterial agents with specificity toward disease-associated oral bacteria.

The following examples are provided to illustrate certain aspects of the present disclosure and are not intended to limit the scope of the disclosure. It will be understood that various modifications and variations can be made based on the present disclosure without departing from the spirit and scope of the disclosure as defined by the appended claims.

### Examples

The search for safe and effective antimicrobial agents for the control and prevention of oral biofilm associated diseases has been ongoing. Clinical trials for oral specific anti-bacterials are costly and often provide inconclusive results. Ex vivo testing of these agents using simple screening approaches has not demonstrated utility, likely due to variability of effects observed even with a single donor.

In this Example, shed oral biofilms, readily obtained from donor saliva, were tested under optimized conditions to evaluate reproducible responses to antibacterial challenges. Reductions in rRNA accumulation in susceptible taxa were used as a measure of antimicrobial activity. While some responses were donor-specific, many bacterial taxa exhibited reproducible susceptibility across multiple donors. When tested at pharmacologic concentrations, two antibiotics, vancomycin and penicillin G, selectively inhibited a subset of Gram-positive bacteria. A natural antibacterial agent, diluted Vaccinium macrocarpon (cranberry) juice, inhibited a range of oral taxa, including Alloprevotella sp. HMT 473, Granulicatella adiacens, Lachnoanaerobaculum umeaense, Leptotrichia sp. HTMT 215, Peptostreptococcus stomatis, Prevotella nanceiensis, Stomatobaculum sp. HMT 097, and Veillonella parvula, and was shown to kill certain species.

The model described in this Example demonstrates a rapid, precise, and reproducible ex vivo method for evaluating and identifying antimicrobial agents with potential clinical utility against the oral biofilm community.

### Results

The goal of the Example was to develop an ex vivo assay to evaluate the effects of antimicrobial agents, in this case, cranberry juice, on oral biofilm. This was done by measuring how this and other agents affect levels of both bacterial rRNA and rDNA. Optimization was performed by identifying reproducible bacterial targets with shed oral biofilm collected and tested on different days from each of the three respective individuals. This shed biofilm assay was used to determine oral bacteria targeted by cranberry juice. The antibiotics penicillin G and vancomycin were used as positive controls.

This study takes advantage of the fact that a substantial fraction of the bacteria in saliva exist as aggregates partially derived from oral biofilm and remain in saliva only transiently (Simon-Soro et al. “Polymicrobial Aggregates in Human Saliva Build the Oral Biofilm.” mBio 2022, 13, e0013122). Human saliva was used as the source for shed oral biofilm because it is a reservoir of microorganisms from all ecological niches in the human oral cavity with long-term stability. In contrast to earlier studies, no attempt was made to isolate, store, or culture the microorganisms, thereby preserving the native community structure of the shed biofilm.

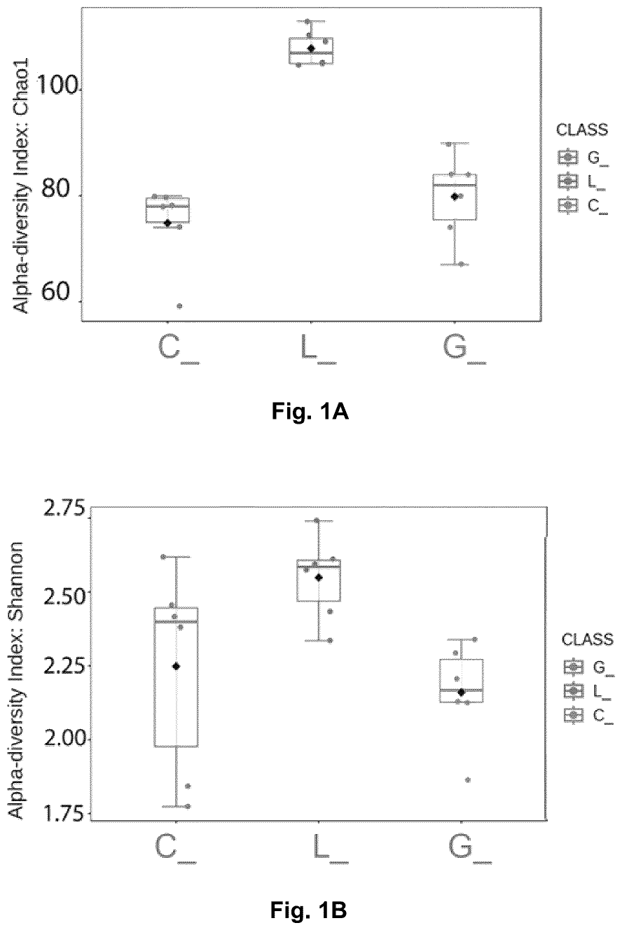

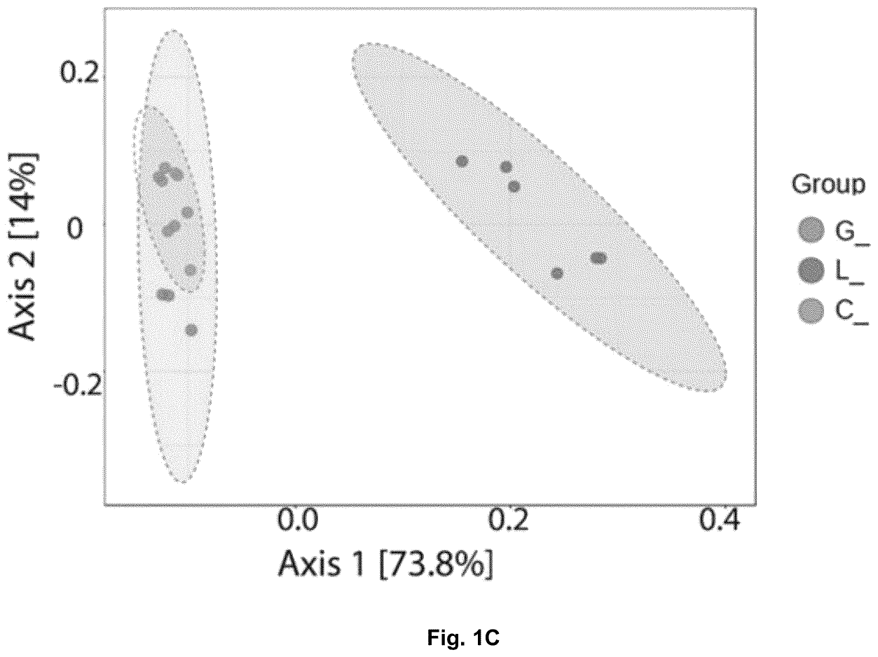

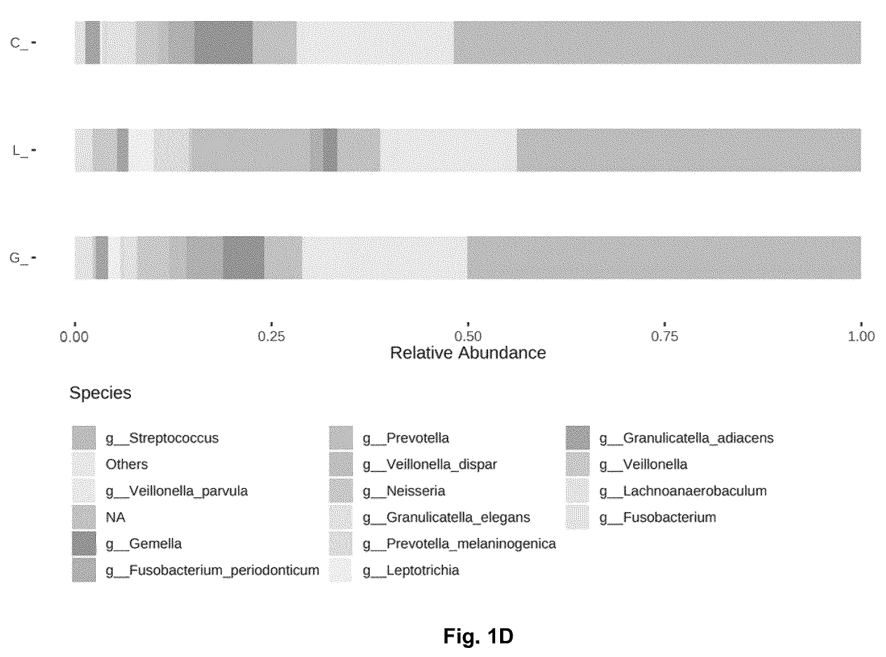

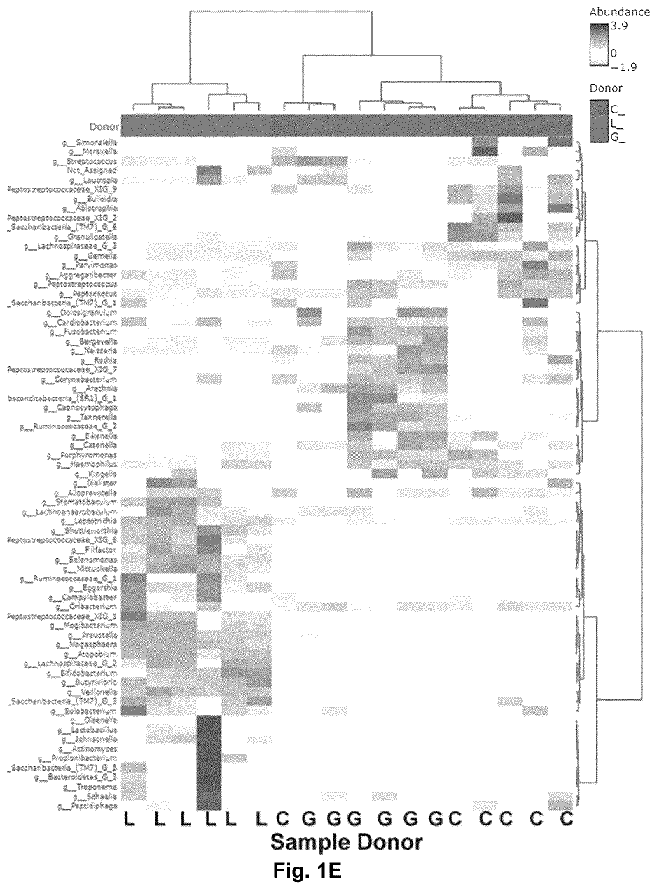

Reproducibility of salivary microbiome transcriptional activity was evaluated by collecting, isolating and testing samples ex vivo over a one-week period. Saliva was isolated at midmorning on day 1, day 3 or 4, and day 7, after a 24-hour period without oral hygiene. Saliva volumes ranged from 15 to 26 mL and were stimulated by chewing 1 g of non-flavored gum base (Wrigley) for 5 min. Three subjects participated in the initial study. Shed biofilm samples were prepared then incubated anaerobically for 4 hours at 37° C. in test medium. RNA was isolated and then subjected to cDNA synthesis and measurement of the 16S rRNA V4 region using next generation DNA sequencing (NGS). Analysis of alpha diversity revealed that the sample sets from the three subjects showed statistically significant between-subject differences. Comparisons of Chao 1 diversity are shown in FIG. 1A, while those for Shannon diversity are shown in FIG. 1B. Analysis of beta diversity, shown in FIG. 1C, revealed that the taxa profiles from each individual grouped together and were again distinct from those of the other two subjects. Unsupervised clustering using the Ward method based on Euclidean Distances identified three distinct groups, with only one sample misclassified to an incorrect donor (FIGS. 1D and 1E).

The same shed biofilm ex vivo isolates, freshly prepared each day, were exposed individually to various agents, namely, antibiotics penicillin G or vancomycin, or 17% cranberry juice, over a 4-hour period. As described below, differences in taxa rRNA expression were detected. An examination of beta diversity (Bray Curtis distances) over the whole group of 45 samples, from the three individuals, tested in the presence of the three different agents on three separate days, was performed using adonis2 to determine the contribution to the diversity by two factors, treatment type and patient ID. Negative controls without any test agent were included as technical replicates. In this test set, 28% of the beta diversity was attributable to donor identity and 30% to treatment type (control, 17% cranberry juice, penicillin G, or vancomycin).

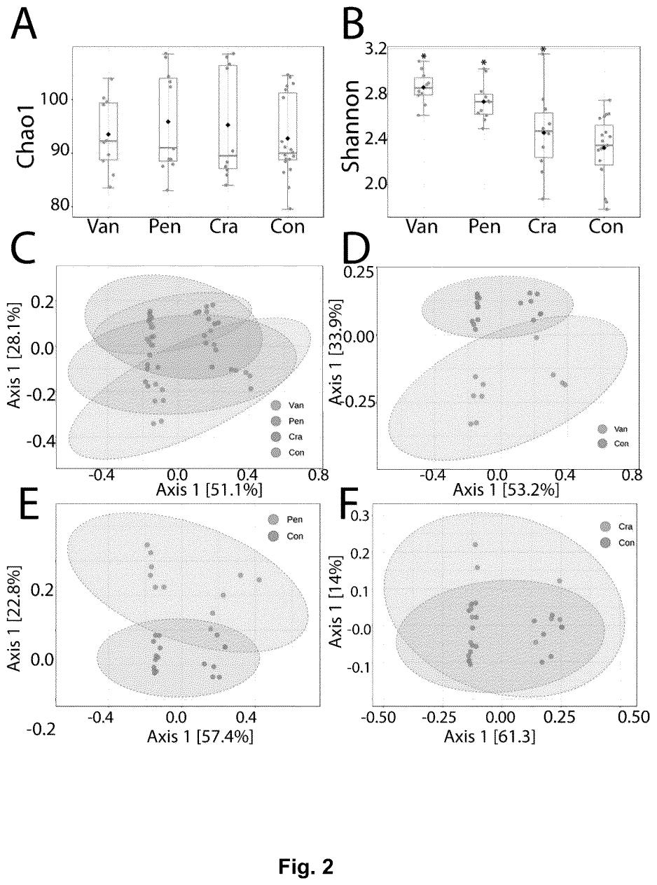

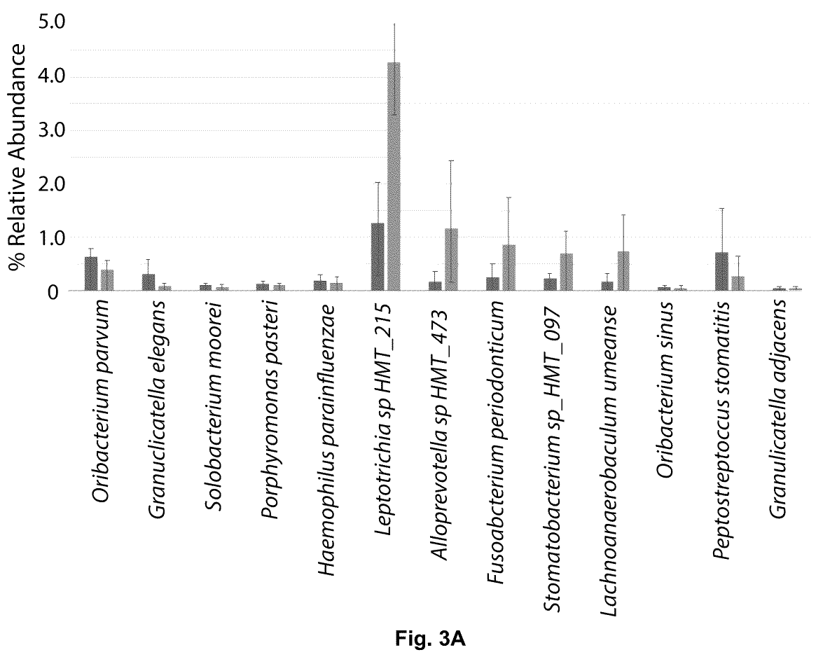

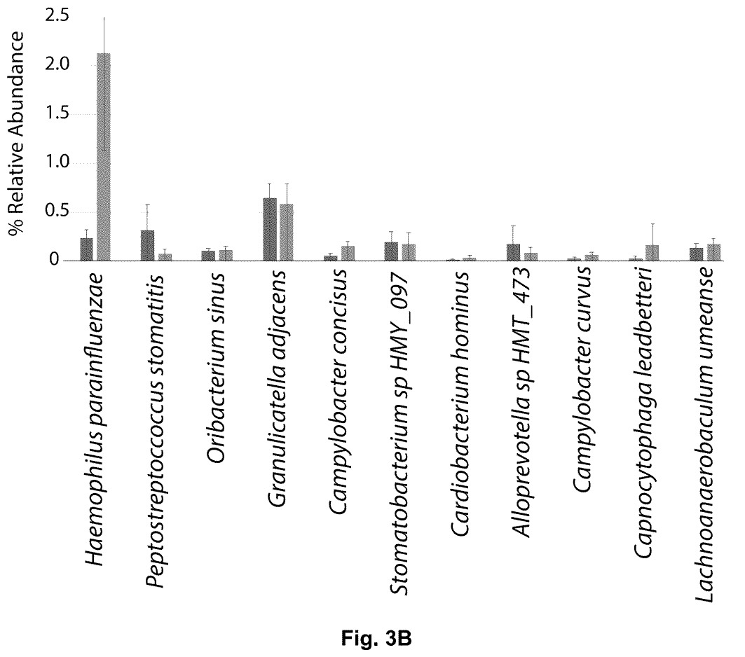

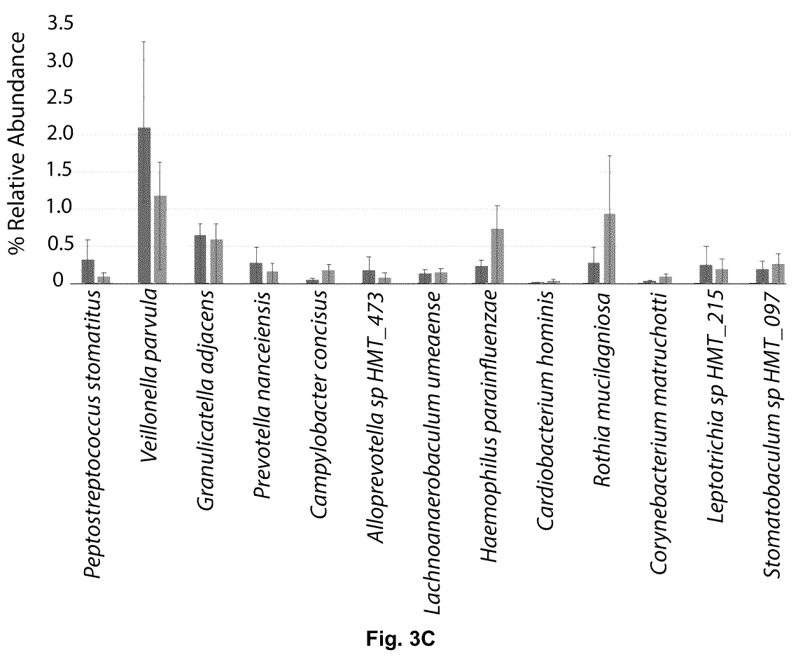

To determine whether exposure to penicillin G induces general changes in taxa gene expression that are consistent across the ex vivo shed biofilm samples from the three subjects, rRNA was analyzed in samples treated with penicillin G at 5 μg/mL, corresponding to the optimal pharmacologic plasma concentration of the drug. This drug, which primarily targets Gram-positive bacteria, would be expected to reduce the relative levels of 16S rRNA in Gram-positive taxa. Shannon alpha diversity, reflecting the richness and evenness of taxa based on 16S rRNA abundance, was observed to be higher after penicillin G exposure (FIG. 2). Beta diversity was also significantly different from that of the mock-treated samples (FIG. 2). Based on DeSeq2 analysis, a broad range of Gram-positive bacteria exhibited lower relative abundance at the genus level. Following penicillin G treatment, samples from all three subjects showed differential abundance in 13 genera compared to the control (Table 1). Of the eight Gram-positive genera identified as differentially abundant, seven displayed reduced relative levels upon penicillin G exposure (Table 1). In contrast, all five Gram-negative genera showed higher relative levels compared to the vehicle-treated shed biofilm samples. At the species level, additional differences relative to the vehicle-treated controls were also observed (FIG. 3B). We note that a subset of alterations in RNA and DNA induced by the agents were donor specific but were reproducible over the three different assays.

TABLE 1
Genera of bacteria with 16S rRNA transcript levels differentially
abundant upon exposure to the test agents in the shed biofilm
ex vivo assay. Values represent mean results of samples
from 3 subjects collected and tested on 3 separate days.
Gram positive genera are shown in bold.

| | | | | |
|---|---|---|---|---|
| Taxon 16S rRNA¹ | log2FC² | lfcSE³ | Pvalues | FDR |

Vancomycin (10 ug/ml)

| | | | | |
|---|---|---|---|---|
| g_{—}Streptococcus | −0.94 | 0.169 | 2.57E−08 | 1.08E−06 |
| g_{—}Peptostreptococcus | −2.50 | 0.471 | 1.09E−07 | 2.28E−06 |
| g_Alloprevotella | 1.71 | 0.338 | 3.97E−07 | 5.55E−06 |
| g_Leptotrichia | 2.03 | 0.456 | 8.02E−06 | 8.42E−05 |
| g_{—}Granulicatella | −1.64 | 0.380 | 1.49E−05 | 1.13E−04 |
| g_{—}Oribacterium | −0.69 | 0.160 | 1.62E−05 | 1.13E−04 |
| g_Fusobacterium | 1.23 | 0.327 | 1.64E−04 | 9.85E−04 |
| g_Haemophilus | 1.21 | 0.335 | 3.21E−04 | 1.69E−03 |
| g_Veillonella | 0.72 | 0.222 | 1.10E−03 | 5.11E−03 |
| g_Porphyromonas | 1.67 | 0.593 | 4.90E−03 | 2.06E−02 |
| g_Aggregatibacter | 1.09 | 0.413 | 8.12E−03 | 3.10E−02 |
| g_Capnocytophaga | 1.23 | 0.515 | 1.69E−02 | 5.91E−02 |
| g_{—}Lachnoanaerobaculum | −0.58 | 0.254 | 2.32E−02 | 7.24E−02 |
| g_Prevotella | 1.05 | 0.471 | 2.61E−02 | 7.24E−02 |
| g_{—}Stomatobaculum | −0.85 | 0.384 | 2.61E−02 | 7.24E−02 |
| g_{—}Solobacterium | −0.97 | 0.439 | 2.76E−02 | 7.24E−02 |

Penicillin G (5 ug/ml)

| | | | | |
|---|---|---|---|---|
| g_Haemophilus | 2.49 | 0.358 | 3.17E−12 | 1.33E−10 |
| g_Aggregatibacter | 2.67 | 0.442 | 1.61E−09 | 3.37E−08 |
| g_Campylobacter | 0.96 | 0.181 | 1.06E−07 | 1.49E−06 |
| g_{—}Peptostreptococcus | −2.63 | 0.504 | 1.85E−07 | 1.95E−06 |
| g_{—}Streptococcus | −0.92 | 0.192 | 1.70E−06 | 1.43E−05 |
| g_{—}Saccharibacteria_(TM7)_{—}G_{—}1 | −1.79 | 0.428 | 2.86E−05 | 2.00E−04 |
| g_Capnocytophaga | 2.05 | 0.535 | 1.24E−04 | 7.44E−04 |
| g_{—}Granulicatella | −1.39 | 0.410 | 7.02E−04 | 3.69E−03 |
| g_{—}Oribacterium | −0.61 | 0.186 | 1.13E−03 | 5.26E−03 |
| g_Cardiobacterium | 1.32 | 0.519 | 1.07E−02 | 4.50E−02 |
| g_{—}Peptostreptococcaceae_{—}XIG_{—}1 | −2.50 | 1.036 | 1.56E−02 | 5.97E−02 |
| g_{—}Stomatobaculum | −0.86 | 0.390 | 2.82E−02 | 9.39E−02 |
| g_{—}Rothia | 0.74 | 0.341 | 2.91E−02 | 9.39E−02 |

Cranberry 17%

| | | | | |
|---|---|---|---|---|
| g_{—}Peptostreptococcus | −2.68 | 0.477 | 2.05E−08 | 8.61E−07 |
| g_Veillonella | −1.37 | 0.262 | 1.85E−07 | 3.88E−06 |
| g_Campylobacter | 0.97 | 0.197 | 8.87E−07 | 1.24E−05 |
| g_Aggregatibacter | 1.09 | 0.350 | 1.88E−03 | 1.98E−02 |
| g_Haemophilus | 0.83 | 0.275 | 2.44E−03 | 2.05E−02 |
| g_{—}Saccharibacteria_(TM7)_{—}G_{—}1 | −1.18 | 0.403 | 3.42E−03 | 2.32E−02 |
| g_{—}Lachnoanaerobaculum | −0.71 | 0.244 | 3.86E−03 | 2.32E−02 |
| g_{—}Cardiobacterium | 1.40 | 0.504 | 5.55E−03 | 2.77E−02 |
| g_{—}Rothia | 0.95 | 0.346 | 6.13E−03 | 2.77E−02 |
| g_Fusobacterium | −0.82 | 0.301 | 6.61E−03 | 2.77E−02 |
| g_{—}Granulicatella | −1.09 | 0.408 | 7.60E−03 | 2.90E−02 |
| g_{—}Schaalia | 0.63 | 0.241 | 8.75E−03 | 3.06E−02 |
| g_Alloprevotella | −0.78 | 0.306 | 1.08E−02 | 3.47E−02 |
| g_{—}Corynebacterium | 0.86 | 0.341 | 1.16E−02 | 3.47E−02 |
| g_{—}Streptococcus | −0.44 | 0.187 | 1.80E−02 | 5.03E−02 |

¹Gram positive taxa are bolded.
²Negative indicates lower levels with antibacterial treatment versus the vehicle control.
³Log fold change standard error.

Vancomycin works through a mechanism distinct from penicillin G to inhibit bacteria but also targets Gram-positive bacteria. Results were generally similar to those for penicillin G-treated shed biofilms. With exposure to vancomycin at 10 μg/mL, corresponding to the optimal pharmacologic serum concentration, the bacteria 16S rRNA profiles from the three subjects were different than vehicle-treated samples. Shannon alpha diversity, reflecting taxa richness and evenness, was higher (p<1.5×10⁶), and Beta diversity was also different. As expected for an antibiotic that targets Gram-positive bacteria, rRNA levels from Gram-positive bacteria tended to be at lower levels versus that for the control. Sixteen genera showed differences in relative abundance over the 4-hour incubation (Table 1). Seven were Gram-positive taxa and all 7 were lower relative to the other taxa. Nine were Gram-negative and all 9 showed higher relative levels versus the controls.

When shed biofilm ex vivo samples from the same three subjects prepared on the same three days were exposed to a 17% mixture of cranberry juice, a large number of bacterial taxa were observed to show levels distinct from those in the vehicle-exposed control. Average alpha diversity between the cranberry-exposed and vehicle-exposed samples was similar. However, beta diversity was substantially different among the two groups as shown in FIG. 2 (p<0.002). DESeq2 identified specific taxa with differentially abundant 16s rRNA transcripts in the cranberry-treated shed biofilm samples compared to the negative controls (FIG. 3C; Table 1). Differentially affected genera included Peptostreptococcus, Veillonella, Campylobacter, Aggregatibacter, Haemophilus, and Fusobacteria, etc., with numerous additional taxa identified at the species level (FIG. 3C; Table 1).

Shed biofilm ex vivo samples from a fourth subject were prepared on three separate days. They were similarly tested with vancomycin, cranberry, or vehicle exposed in triplicate to independently validate the preceding findings (Table 2). Independent 16S rRNA amplicon sequencing of the RNA revealed substantial overlap with the results obtained from the first three subjects. Among the 16 genera differentially represented following vancomycin treatment, five (Peptostreptococcus, Alloprevotella, Granulicatella, Haemophilus, and Leptotrichia) exhibited changes consistent with those observed previously (Tables 1 and 2). For cranberry exposure, 7 out of the 16 genera were similarly differentially abundant, including Peptostreptococcus, Veillonella, Campylobacter, Aggregatibacter Haemophilus, Rothia, and Alloprevotella (Tables 1 and 2).

TABLE 2
Genera of bacteria with 16S rRNA transcript levels differentially abundant
upon exposure to the test agents in ex vivo shed saliva bacteria aggregates
obtained from a fourth independent donor. Each treatment was performed in
triplicate using saliva samples collected and tested on three separate days.

| | | | | |
|---|---|---|---|---|
| Taxon 16S rRNA | log2FC | lfcSE* | Pvalues | FDR |

Vancomycin (10 ug/ml)

| | | | | |
|---|---|---|---|---|
| g_Mogibacterium | −2.3464 | 0.34514 | 1.06E−11 | 4.66E−10 |
| g_Granulicatella | −1.4581 | 0.3181 | 4.57E−06 | 0.00010051 |
| g_Enterococcus | −7.6275 | 1.7015 | 7.37E−06 | 0.00010804 |
| g_Peptostreptococcaceae_XIG_1 | −1.8205 | 0.43519 | 2.87E−05 | 0.00031604 |
| g_Leptotrichia | 1.9136 | 0.5552 | 0.00056747 | 0.0049937 |
| g_Solobacterium | −1.1498 | 0.34035 | 0.00072947 | 0.0053494 |
| g_Haemophilus | 1.9788 | 0.62986 | 0.0016799 | 0.010559 |
| g_Alloprevotella | 1.0953 | 0.37409 | 0.0034125 | 0.018769 |
| g_Lachnoanaerobaculum | −0.67861 | 0.26318 | 0.0099231 | 0.048513 |
| g_Selenomonas | 1.1164 | 0.50275 | 0.026378 | 0.11505 |
| g_Peptostreptococcus | −2.3609 | 1.0851 | 0.029572 | 0.11505 |
| g_Saccharibacteria_(TM7)_G_1 | −1.5579 | 0.72385 | 0.031378 | 0.11505 |
| g_Rothia | 1.1462 | 0.55434 | 0.038666 | 0.13087 |

Cranberry 17%

| | | | | |
|---|---|---|---|---|
| g_Mogibacterium | −1.6715 | 0.24729 | 1.3866E−11 | 6.933E−10 |
| g_Veillonella | −1.2295 | 0.2105 | 5.1843E−09 | 1.2961E−07 |
| g_Prevotella | −0.90912 | 0.22477 | 5.2405E−05 | 0.00078814 |
| g_Campylobacter | 1.0475 | 0.2618 | 6.3051E−05 | 0.00078814 |
| g_Peptostreptococcus | −2.5055 | 0.66209 | 0.00015421 | 0.0015421 |
| g_Solobacterium | −1.1526 | 0.33601 | 0.00060295 | 0.0049416 |
| g_Haemophilus | 1.4055 | 0.41426 | 0.00069182 | 0.0049416 |
| g_Alloprevotella | 1.0731 | 0.32445 | 0.00094159 | 0.005885 |
| g_Porphyromonas | −1.2029 | 0.38464 | 0.0017639 | 0.0097993 |
| g_Neisseria | 0.84914 | 0.30045 | 0.0047097 | 0.022276 |
| g_Peptostreptococcaceae_XIG_1 | −1.1152 | 0.39875 | 0.0051606 | 0.022276 |
| g_Oribacterium | −0.83563 | 0.3 | 0.0053463 | 0.022276 |
| g_Streptococcus | −0.62665 | 0.26722 | 0.019023 | 0.073166 |
| g_Rothia | 0.99022 | 0.42969 | 0.021194 | 0.075692 |
| g_Aggregatibacter | 1.4331 | 0.67222 | 0.033013 | 0.11004 |
| g_Actinomyces | 1.0346 | 0.49786 | 0.037703 | 0.11782 |

*lfcSE is standard error of the mean of the log fold change.

To determine whether the changes in rRNA expression in shed biofilm ex vivo samples treated with vancomycin, penicillin G, or cranberry were reflected in differences in relative 16S rDNA levels, both DNA and rRNA were isolated from samples exposed to the three agents and the control. Samples were from the 4 subjects, with an N of 21 for each agent versus the controls. Table 3 lists the taxa differentially abundant on the level of 16S rRNA after exposure to each agent. This analysis was based on a smaller subset of samples and used an abundance measure distinct from that used for Table 1 and FIGS. 3A-3C; therefore, some divergence from those earlier results was expected. Table 4 lists the taxa that were differentially abundant at the level of 16S rDNA (the 16S rRNA gene). Overall, a greater number of taxa showed differential abundances of 16S rRNA transcripts than 16S rDNA after exposure to each agent versus the control (Tables 1 and 3).

TABLE 3
MaAsLin2 analysis identifying taxa differentially abundant at the RNA
level in the shed biofilm ex vivo assay following exposure to the test
agents, based on a subset of test samples from 4 subjects. 16S rRNA
levels were compared to those of the vehicle-exposed samples.

| | | | | | | |
|---|---|---|---|---|---|---|
| Taxon 16S rRNA | coef | stderr | N | N.not.0 | pval | qval |

Vancomycin

| | | | | | | |
|---|---|---|---|---|---|---|
| g_Granulicatella.s_adiacens | −1.229 | 0.208 | 21 | 21 | 0.0000 | 0.0016 |
| f_Fusobacteriaceae.g_Fusobacterium._{—} | 0.802 | 0.148 | 21 | 21 | 0.0000 | 0.0020 |
| f_Leptotrichiaceae.g_Leptotrichia._{—} | 1.619 | 0.391 | 21 | 21 | 0.0006 | 0.0226 |
| f_Streptococcaceae.g_Streptococcus._{—} | −0.885 | 0.227 | 21 | 21 | 0.0010 | 0.0290 |

Penicillin G

| | | | | | | |
|---|---|---|---|---|---|---|
| f_Pasteurellaceae.g_Haemophilus._{—} | 1.954 | 0.417 | 20 | 20 | 0.0002 | 0.0204 |
| g_Haemophilus.s_parainfluenzae | 1.812 | 0.464 | 20 | 20 | 0.0010 | 0.0566 |

Cranberry

| | | | | | | |
|---|---|---|---|---|---|---|
| g_Campylobacter.s_curvus | 1.378 | 0.215 | 21 | 21 | 0.0000 | 0.0006 |
| f_Pasteurellaceae.g_Haemophilus._{—} | 1.498 | 0.284 | 21 | 21 | 0.0000 | 0.0025 |
| g_Rothia.s_dentocariosa | 1.259 | 0.266 | 21 | 21 | 0.0002 | 0.0048 |
| f_Neisseriaceae.g_Kingella.s_oralis | 1.756 | 0.370 | 21 | 11 | 0.0002 | 0.0048 |
| g_Haemophilus.s_parainfluenzae | 1.241 | 0.270 | 21 | 21 | 0.0002 | 0.0051 |
| f_Campylobacteraceae.g_Campylobacter._{—} | 1.459 | 0.322 | 21 | 14 | 0.0003 | 0.0051 |
| f_Pasteurellaceae.g_Aggregatibacter._{—} | 1.615 | 0.379 | 21 | 21 | 0.0005 | 0.0067 |
| f_Actinomycetaceae.g_Actinomyces._{—} | 1.163 | 0.269 | 21 | 21 | 0.0004 | 0.0067 |
| g_Rothia.s_mucilaginosa | 1.057 | 0.267 | 21 | 21 | 0.0009 | 0.0120 |
| g_Campylobacter.s_concisus | 1.194 | 0.309 | 21 | 21 | 0.0011 | 0.0131 |
| g_Corynebacterium.s_matruchotii | 0.920 | 0.272 | 21 | 19 | 0.0033 | 0.0340 |
| g_Cardiobacterium.s_hominis | 1.392 | 0.415 | 21 | 16 | 0.0035 | 0.0340 |
| g_Actinomyces.s_graevenitzii | 1.101 | 0.350 | 21 | 21 | 0.0054 | 0.0479 |
| g_Peptostreptococcus.s_stomatis | −1.448 | 0.499 | 21 | 21 | 0.0096 | 0.0792 |
| g_Granulicatella.s_adiacens | −0.633 | 0.231 | 21 | 21 | 0.0136 | 0.1050 |

TABLE 4
MaAsLin2 analysis identifying taxa differentially abundant at the DNA
level in the shed biofilm ex vivo assay following exposure to the
tested agents, based on a subset of test samples from 4 subjects.

| | | | | | | |
|---|---|---|---|---|---|---|
| Taxon 16S rRNA Gene (DNA) | coef | stderr | N | N.not.0 | pval | qval |

Penicillin G

None

Vancomycin

None

Cranberry

| | | | | | | |
|---|---|---|---|---|---|---|
| f_Streptococcaceae.g_Streptococcus._{—} | −0.9744 | 0.1825 | 21 | 21 | 0.0000 | 0.0023 |
| g_Veillonella.s_parvula | −0.7490 | 0.1349 | 21 | 21 | 0.0000 | 0.0023 |
| g_Rothia.s_dentocariosa | 1.2107 | 0.3333 | 21 | 21 | 0.0018 | 0.0738 |

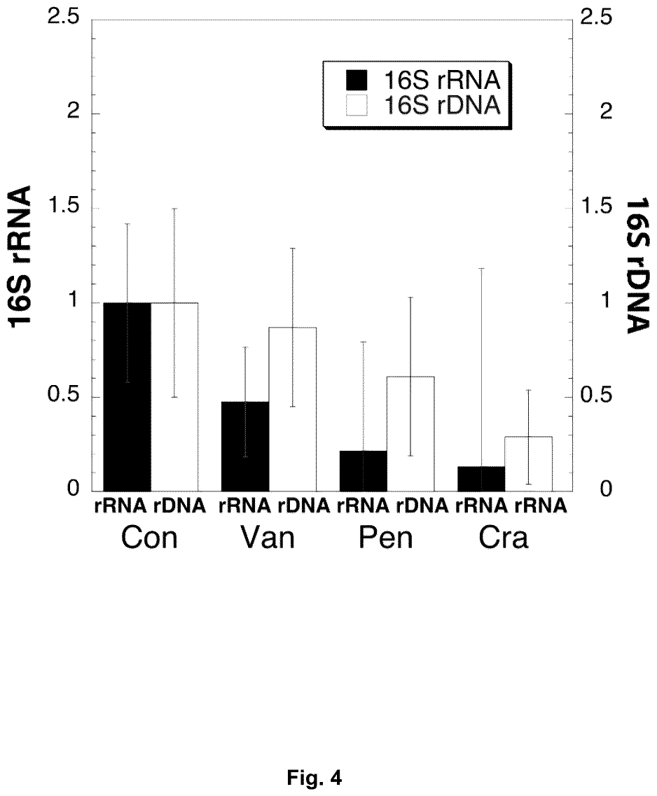

An important question was whether the absolute levels of bulk 16S rDNA and 16S rRNA transcripts differed with the three different treatments versus the control. Analysis of total relative yield of 16S rDNA in each sample based on PCR revealed no differences in vancomycin-treated samples versus controls (0.81× the control; p<0.31); lower levels in penicillin G-treated samples (0.53× the control), trending toward significance (p<0.065); and markedly reduced levels in cranberry-treated samples (0.14× the control; p<0.002) (FIG. 4). When the same analysis was performed at the 16S rRNA (RNA) level all treatments resulted in lower levels of 16S rRNA transcripts with exposure to vancomycin (0.33×; p<0.001), penicillin G (0.16×; p<0.004), and cranberry (0.28×; p<0.038).

An analysis (N=26) of total 16S rDNA levels in shed biofilm samples at baseline and then after the 4-hour incubation with 17% cranberry extract revealed a decrease in rDNA levels (0.43×; p<0.0203). In contrast, shed biofilm samples incubated with penicillin G showed minimal change (1.6×; p<0.12), while those incubated with vancomycin exhibited a trend toward increased levels (2.4×; p<0.0658) (Table 5, right). A similar analysis of total 16S rRNA levels (N=25) was performed under identical assay conditions, comparing shed biofilm samples placed in test media immediately terminated by adding RNA preservative to samples incubated for 4 hours in test media and 17% cranberry juice, vancomycin, or penicillin G prior to termination (Table 5, left). No significant changes in 16S rRNA levels were observed.

TABLE 5
Comparison of relative levels of 16S rRNA and
rDNA in shed biofilm ex vivo samples before
and after 4-hour exposure to the test agents.

| | 16S rRNA | p value | 16S rDNA | p value |
|---|---|---|---|---|
| Shed Biofilm Assay | Level | vs 0 HR | Gene Level | vs 0 HR |
| Vehicle + 0 HR Incub. | 1.0 ± 0.50 | | 1.0 ± 0.91 | |
| Vancomycin + 4 HR | 2.2 ± 1.24 | 0.012 | 2.43 ± 1.44 | 0.0658 |
| Incub. | | | | |
| Penicillin G + 4 HR | 0.97 ± 0.58 | 0.67 | 1.57 ± 0.76 | 0.121 |
| Incub. | | | | |
| Cranberry + 4 HR Incub. | 0.63 ± 0.53 | 0.67 | 0.43 ± 0.38 | 0.0203 |

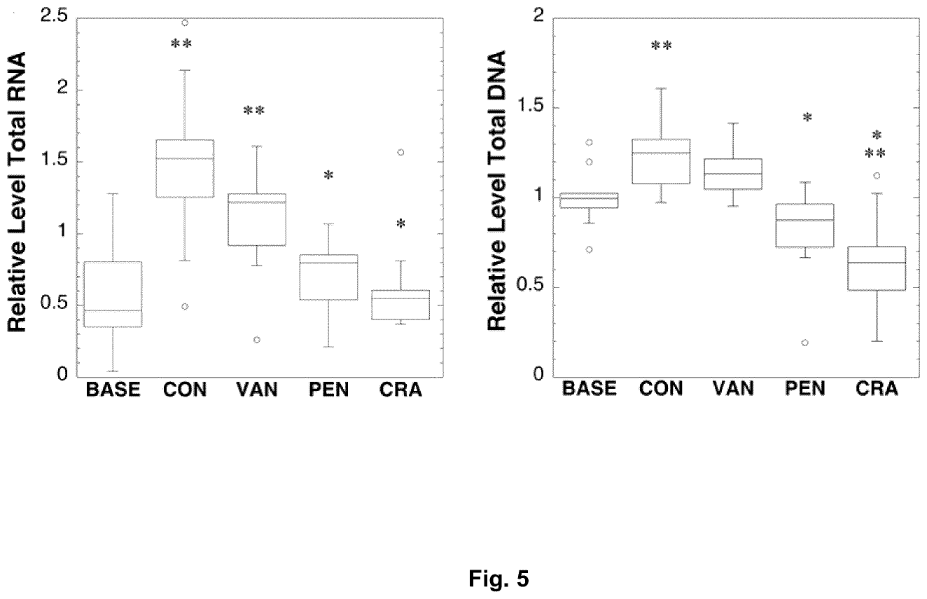

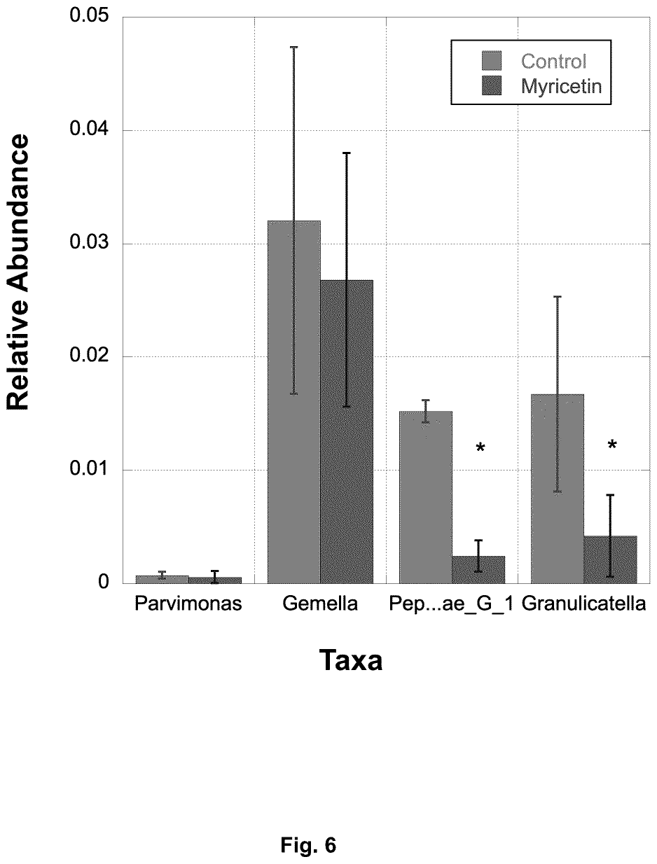

Levels of bulk RNA and DNA recovered from the assayed samples revealed similar results to those with 16S rRNA and DNA for cranberry and vancomycin, but not for penicillin G. Both penicillin G and 17% cranberry juice treatments over the 4 hours resulted in a decrease in total DNA versus the baseline (Base) shed saliva biofilm at the start of the assay (FIG. 5).

Additionally, we examined whether the shed biofilm assay can detect antimicrobial effects during a shorter incubation period. Myricetin at a concentration of 1 mM was shown to target specific oral bacterial taxa in both the 4-hour and 1-hour ex vivo shed oral biofilm assays. As shown in Table 6, DeSeq2 analysis identified genera with 16S rRNA transcript levels that were differentially abundant after 4-hour exposure. These same taxa were then evaluated in a shortened 1-hour assay, and FIG. 6 demonstrates that 2 of the 4 taxa remained differentially abundant compared to the control (p<0.05). This representative test of an additional agent demonstrates that the assay is capable of detecting antimicrobial effects within 1 hour, even when measuring 16S rRNA. Because mRNA has a faster turnover rate than 16S rRNA, the assay would be expected to detect the effects of an agent targeting a specific microbial group in a similar 1-hour or shorter incubation when mRNA markers are used.

TABLE 6
Genera of bacteria with 16S rRNA transcript levels differentially
abundant (decreased) upon 4 hour exposure to 1 mM myricetin in
the shed biofilm ex vivo assay as determined by DeSeq2 analysis.

| Taxa | log2FC¹ | lfcSE ² | Pvalues | FDR³ |
|---|---|---|---|---|
| g_Parvimonas | 2.8927 | 0.57962 | 6.01E−07 | 4.57E−05 |
| g_Gemella | 0.83729 | 0.2231 | 0.00017472 | 0.0066394 |
| g_Peptostreptococcaceae_G_1 | 1.7003 | 0.50262 | 0.00071709 | 0.013625 |
| g_Granulicatella | 1.251 | 0.41749 | 0.002732 | 0.041527 |

¹log2FC is logarithmic value of the fold change in level versus the mock treated control incubation.
² lfcSE standard error of the log fold change estimates.
³FDR—false discovery rate.

### Discussion

Changes in rRNA with the Agents

Differences in levels of rRNA in response to agents known to affect bacteria were observed in this ex vivo assay of shed oral biofilm. Like oral biofilms, salivary bacterial aggregates are thought to share the properties of relative antibiotic resistance, resistance to immune regulation, and protection from chemicals. Relatively higher and lower levels of specific 16S rRNAs from various taxa were observed after exposure to each of the three agents versus the control. The results were unexpected because changes indicative of loss of ribosomal RNA might be minimal during the 4-hour period, as rRNA can have turnover rates over days even after cell death (see Maivali et al. “When stable RNA becomes unstable: the degradation of ribosomes in bacteria and beyond.” Biol Chem 2013, 394, 845-855; Deutscher. “Degradation of stable RNA in bacteria.” J Biol Chem 2003, 278, 45041-45044; and Schostag et al. “Low Turnover of Soil Bacterial rRNA at Low Temperatures.” Front Microbiol 2020, 11, 962). On the other hand, rapid increases in rRNA levels versus control are known to occur in responsive taxa as they increase in activity and protein synthesis levels go up. For the penicillin G- and vancomycin-treated samples, the lower 16S rRNA levels observed for certain species relative to control were likely due to the absence of an increase rather than an absolute decrease. At present, it remains unclear whether taxa showing lower 16S rRNA levels (Table 1; FIGS. 3A-3C) represent true decreases or bacteria that fail to be stimulated in the presence of inhibitory agents. If the latter is the case, the assay may preferentially measure those taxa within the community that remain active or initiate proliferation under ex vivo conditions. It is notable that a large number of taxa showed differential 16S rRNA levels in response to each of the three agents (FIGS. 3A-3C), suggesting the assay effectively interrogates a broad range of bacterial species.

### Reproducibility of Results

It was initially unclear how oral bacteria isolated in bulk within their native communities would respond to the same treatment when tested on different days or at different times of day. In vivo studies in humans suggest that oral bacteria DNA levels do not change greatly over time, but bacteria rRNA levels may show more variability. However, the effects of cranberry extract, penicillin G, or vancomycin on oral bacteria rRNA levels in vivo have not been characterized. In addition, variability in the characteristics of ex vivo samples collected on different days could contribute to reduced reproducibility. This could be due to differences in nutrients, host factors, or cells that are trapped in the matrix of the ex vivo material and not completely removed during sample preparation.

Despite these potential sources of variation, rRNA analysis demonstrated high reproducibility among three samples collected over a one-week period from a single individual and incubated as negative controls in test media for 4 hours (FIGS. 1A-1E).

Moreover, the high degree of concordance in rRNA responses to each tested agent among samples from different subjects was unexpected, given the subject-specific differences in baseline 16S rRNA taxa profiles (FIGS. 1A-1E). Differences in taxa rRNA abundance in response to the tested agents were highly similar among different donors (FIG. 2). Saliva biofilm samples from a fourth individual, tested on three separate days, showed substantial overlap in the taxa that became more abundant depending on the agent applied compared with the controls (Tables 1 and 2). These findings support the robustness of the ex vivo shed biofilm assay for detecting consistent bacterial responses and common antimicrobial targets across different individuals.

### Selectivity of Controls

The antibiotics used as controls in this study, penicillin G and vancomycin, both target cell wall synthesis though they have distinct mechanisms. They target Gram-positive bacteria with thick cell walls unprotected by a lipid membrane. While vancomycin is a glycopeptide that binds directly to cell wall peptidoglycan to prevent crosslinking and leads to a faulty cell wall, penicillin G is a beta lactam molecule that binds to and inhibits enzymes responsible for forming the cell wall crosslinks. Because both antibiotics generally cannot cross the outer membrane of Gram-negative bacteria, their activity against those taxa is limited. In the ex vivo shed biofilm experiments, both drugs selectively reduced the relative abundance of Gram-positive bacteria when taxa were analyzed at the genus level (Table 1). At the species level, several Gram-negative bacteria were also observed at lower relative abundance, which may represent either direct or indirect effects of the antibiotics. Such changes could result from the disruption of mutualistic relationships between Gram-positive and Gram-negative species, although it is also possible that certain Gram-negative taxa, capable of drug uptake, are directly susceptible to these agents.

Effects of Agents on 16S rDNA

The focus of this study was on rRNA as it was thought to show more rapid changes in response to stimulation or inhibition than DNA. However, during the 4-hour incubation period, some bacteria may have proliferated while others may have undergone cell death, potentially leading to detectable loss of genomic DNA. Indeed, even over this short period, measurable differences in total 16S rDNA were observed in samples exposed to cranberry extract compared with vehicle-treated controls (FIG. 4).

16S rDNA levels of vancomycin-treated samples were not significantly different from those of controls, while penicillin G treatment trended toward lower levels of bacteria DNA (0.52×; p<0.065), and cranberry exposure resulted in markedly reduced 16S rDNA (gene) levels (0.13×; p<0.0005). Given the reduced total rRNA and rDNA observed with 17% cranberry treatment versus the vehicle-treated shed biofilm, we sought to determine if the reduction was an actual DNA loss during incubation or simply a block of bacterial DNA production.

Comparisons of samples before and after 4-hour exposure to 17% cranberry juice showed a statistically significant decrease in 16S rDNA levels (38% compared to the total level of 16S rDNA at assay start; p<0.044) (Table 5). When effects on bulk DNA were measured, both penicillin G and 17% cranberry treatment induced reductions in DNA levels over the 4-hour incubation (FIG. 5). The basis for the discrepancy between changes in 16S rDNA and total DNA following penicillin G treatment remains unclear. The rapid decline in bacterial DNA was unexpected given previous reports of slower genomic DNA degradation kinetics (see e.g., Masters et al. “Effect of stress treatments on the detection of Listeria monocytogenes and enterotoxigenic Escherichia coli by the polymerase chain reaction.” J Appl Bacteriol 1994, 77, 73-79; Dupray et al. “Salmonella DNA persistence in natural seawaters using PCR analysis.” J Appl Microbiol 1997, 82, 507-510; and Josephson et al. “Polymerase chain reaction detection of nonviable bacterial pathogens.” Appl Environ Microbiol 1993, 59, 3513-3515). These findings highlight the strong disruptive capabilities of the 17% cranberry extract.

Effects of Agents on 16S rDNA Vs 16S rRNA Transcripts

It is well established that in various microbiomes, RNA and DNA levels attributed to specific taxa often do not agree, as DNA abundance reflects the total number of bacterial cells (both viable and non-viable) and RNA levels are more indicative of the state of the bacteria population. The rationale of the present study was that the ex vivo exposure of shed oral biofilms to various agents would induce measurable changes in RNA levels more rapidly than in DNA, even for rRNA transcripts. This provides a means to assess antimicrobial activity without the extended incubation periods typically required for DNA-based bactericidal measurements. Although RNA changes may, in some settings, parallel changes in DNA due to proliferation or cell loss, analysis of the present data indicates that the RNA-level responses are distinct.

Table 3 represents a head-on comparison of RNA and DNA differences for the same 16S rRNA genes and transcripts in the same ex vivo samples treated with vancomycin, penicillin G, or cranberry, revealed minimal overlap between RNA- and DNA-level responses. For this experiment, only samples available as both RNA and DNA were included, and batch correction was applied using the MaAsLin2 method. These findings are not directly comparable to those shown in FIGS. 3A-3C, which were derived from a larger sample set. In this subset analysis, a greater number of taxa exhibited differential abundance at the RNA level than at the DNA level for all three agents tested (Tables 3 and 4). The comparatively lower sensitivity of the DNA-based analysis limited the ability to assess correspondence between the 16S rRNA and 16S rDNA profiles. Collectively, the data support that rRNA measurements provide a more sensitive and rapid indicator of taxa affected by antimicrobial agents at early timepoints. The consistent reduction in total 16S rRNA following exposure to all three agents versus vehicle-treated controls further demonstrates the responsiveness of the rRNA assay.

Head-to-head comparisons of RNA and DNA levels from the same samples may vary in accuracy depending on the methods used for bacterial lysis and nucleic acid purification. Additional studies may further characterize the earliest timepoints at which changes in rRNA can be detected in the present assay. Comparisons between the effects of antimicrobial agents in vivo and those observed in ex vivo shed biofilm assays may also help illustrate how closely the assay reflects in vivo responses. Recent in vivo studies in adults and children have demonstrated that rinsing orally with 100% pure cranberry juice inhibited oral plaque regrowth and reduced metabolic activity including acid production. This supports the concept that the ex vivo assay described herein should detect measurable effects of cranberry exposure on specific bacterial taxa. For example, earlier in vitro studies demonstrated reductions in Streptococcus mutans levels in some oral compartments after 4 or 6 weeks of rinsing with cranberry extract (Weiss et al. “A high molecular mass cranberry constituent reduces mutans streptococci level in saliva and inhibits in vitro adhesion to hydroxyapatite.” FEMS Microbiol Lett 2004, 232, 89-92; and Gupta et al. “Effect of high-molecular-weight component of Cranberry on plaque and salivary Streptococcus mutans counts in children: an in vivo study.” J Indian Soc Pedod Prev Dent 2015, 33, 128-133), consistent with the reduction in Streptococcus genus observed in the present study (FIG. 3C). In the current work, S. mutans were at or near the detection limit in all except one subject sample, limiting the ability to assess the effect of cranberry on that species. No measurable reduction in S. mutans was observed, likely reflecting the low baseline levels in the caries-free donors who provided samples. Other studies have shown that Actinomyces naeslundii and to a lesser extent S. mutans were inhibited by cranberry in planktonic culture, but Lactobacillus paracresis was unaffected. Another study reported that short-term exposure of planktonic and biofilm cultures to 100% cranberry juice markedly inhibited Veillonella parvula and A. naeslundii. In the ex vivo assay described herein, both V. parvula and A. naeslundii were queried. Only V. parvula exhibited a lower abundance in cranberry-treated shed biofilm samples (FIG. 3C). Application of full-length 16S rRNA amplicon sequencing in future work may further clarify the relationship between results from traditional in vitro systems and those obtained using the ex vivo assay.

It may also be informative to evaluate how closely the present system reflects the in vivo environment in which individuals rinse with 100% cranberry juice. The current assay used 17% cranberry juice with a 4-hour incubation period, which may not fully replicate the physiological exposure conditions in vivo. Further evaluation may help clarify the extent to which anaerobically tested shed biofilms mirror the responses of oral biofilms in vivo.

### CONCLUSION

The shed biofilm ex vivo assay demonstrated reproducibility, as oral bacterial communities isolated on different days produced similar 16S rRNA profiles following incubation (FIGS. 1A-1E). It also revealed that both positive control agents, penicillin G and vancomycin, led to reduced steady-state levels of 16S rRNA predominantly from Gram-positive genera (FIGS. 3A and 3B), consistent with their known inhibitory effects on Gram-positives. In contrast, treatment with diluted cranberry extract caused reduced 16S rRNA levels across a broad range of bacterial taxa versus the control. Comparison of pre- and post-incubation samples with the diluted cranberry extract also indicated reduced 16S rDNA levels, suggesting that active killing occurred. This was a surprising result as bacterial DNA is typically considered stable even in dead cells. The observed reduction in Streptococcus abundance in the shed biofilm assay aligns with previous in vivo studies showing decreased Streptococcus levels after oral exposure to cranberry formulations. In follow-up studies, application of mRNA analysis may provide deeper insight into the mechanisms of how the test agents affect bacterial molecular pathways. It is important to recognize that oral diseases, whether initiated by a single pathogenic taxon or by changes in multiple taxa, ultimately arise from community-level changes that promote a dysbiotic microbial state. Therefore, agents identified through the shed biofilm assay that support or restore a bacterial community structure associated with oral health, rather than disease, would be of particular therapeutic interest.

### Materials and Methods

Materials: penicillin G was prepared at 12.5 mg/mL in water and vancomycin was dissolved in DMSO at 2.5 mg/mL; final concentrations were 5 μg/mL and 10 μg/mL, respectively. Pure unsweetened cranberry juice (Ocean Spray) was used at 16.7% final concentration.

Ethics approval was by the Institutional Review Board I at the University of Illinois Chicago (IRB protocol #2016-0696). Verbal and written informed consent were obtained by all participants prior to saliva collection. All experiments were performed in accordance with relevant guidelines and regulations.

### Sample Collection, DNA/RNA Extraction, and Sequencing

Participants in overall good health without overt systematic disease and not taking antibiotics over the previous three months, were directed to refrain from oral hygiene for 24 hours prior to supplying stimulated saliva accumulated from chewing 1 g of non-flavored gum base (Wrigley) for 5 minutes. Saliva was put on ice, centrifuged at 5500×G for 5 min at 4° C., then washed with transport medium, resuspended in PBS and adjusted to OD_{600 nm}=1.0, then centrifuged and resuspended in same volume of modified SHI medium supplemented with 2% BSA and 0.1% glucose. The modified SHI medium, which lacks sheep blood and sucrose, had the following composition: 10 g/L proteose peptone (Fisher, BP1420-500); 5.0 g/L trypticase peptone (Fischer, BP 1421-500); 5.0 g/L yeast extract (BD Bacto, 212750); 2.5 g/L KCl (Fisher, P217-500); 5 mg/L hemin (Sigma, 51280); 1 mg/L Vitamin K (Sigma, M5625-25G); 0.06 g/L urea (Fisher, U15-3), 0.174 g/L arginine (Fisher, BP370-100); 2.5 g/L mucin (Sigma, M1778-10G); and 10 mg/L N-acetylmuramic acid (Sigma, A3008-100MG).

Salivary bacterial suspension (400 μL) was then added into 2 mL polypropylene centrifuge tubes containing test agent (in total 100 μL volume) and incubated anaerobically at 37° C. for 4 hours in an anaerobic chamber (Forma) filled with anaerobic gas (5% CO₂, 10% H₂, 85% N₂). Water was used as a control and all test samples had end concentrations of 0.40% DMSO including controls with no agent.

Post incubation with various agents under anaerobic conditions; 2 volumes RNA Protect Cell (Qiagen) was added to each tube to stop reactions and preserve nucleic acid. Samples were placed on ice for 30 minutes. Samples were then centrifuged for 10 min at 5,500× G, and supernatant removed. Pellets were dissolved in guanidium thiocyanate solution (GTC, 4 M guanidinium thiocyanate, 25 mM sodium citrate, pH 7.0), 0.5% (w/v) N-laurosylsarcosine (Sarkosyl), and 0.1 M 2-mercaptoethanol, or RLT from Qiagen, followed by homogenization with 0.5 and 0.1 mm glass-zirconium beads in the Mini-Bead Beater 3× at 1 min each. Acid phenol extraction was followed by silica-based purification using Zymo Research RNA Clean and Concentrator-5. RNA samples were all exposed to Turbo DNase at 1 unit/mL for 30 min at 37° C. prior to repurification. cDNA synthesis was carried out using random sequence hexamers and Superscript III (Invitrogen, Thermo Fisher Scientific) using 1% of each sample harvested from the original bacterial pellets, then subjected to amplicon sequencing similar to DNA samples as described below.

DNA was extracted from a 150 μL aliquot of lysed bacteria in GTC at −80° C. which was left over from mechanical lysis done for RNA extraction. After addition of 2 volumes Bashing Buffer, the Quick-DNA Fungal/Bacteria Miniprep Kit (Zymo Research) was used to purify the DNA. The V4 variable region of bacterial 16S rRNA genes was amplified using the primer set

CS1_515F:
(SEQ ID NO: 1)
ACACTGACGACATGGTTCTACAGTGTGYCAGCMGCCGCGGTAA
CS2_806R:
(SEQ ID NO: 2)
TACGGTAGCAGAGACTTGGTCTCCGGACTACNVGGGTWTCTAAT

followed at the Rush University Genomics and Microbiome Core by a second PCR amplification when sample specific barcodes were added followed by cleanup and sequencing. Sequencing was performed on the Illumina MiniSeq2 at 150 cycles (Illumina, Inc, San Diego, CA, USA). Negative controls were samples that started with H₂O instead of saliva DNA. Additional controls were technical replicates of several assays.

For bulk RNA and DNA measurements either Nanodrop was used to measure A260 of nucleic acids, or a Qubit was used with Biotium AccuGreen™ High Sensitivity dsDNA and AccuBlue® Broad Range RNA Quantitation Kits. For measurement of bulk 16S rRNA and rDNA, a PCR-based method was used (Nadkarni, 2002).

### Microbial Community Analysis

For taxa assignment and measurement, forward sequence reads from the FASTQ files were analyzed using the software package QIIME2 (v2023.5). Sequences were all at average quality score of 25 without trimming and were used as a size of 151. DADA2-plugin in QIIME2 was used to denoise the sequence and generate feature data and feature tables for the dataset of DNA sequences. Taxonomy assignment was done by classify-consensus-blast function using the Blast+ consensus taxonomy classifier to determine 98% match identity of the query sequences to the Human Oral Microbiome Database (v15.22). On average there were 52,815 reads per sample with the minimum 17,243 for cDNA amplicon sequencing and average 67,417 reads per samples with the minimum 38,208 reads for amplicon DNA sequencing.

Taxa that had fewer than 2 reads in 80% of samples were eliminated. Alpha and beta diversity were determined on the feature level using MicrobiomeAnalyst. For the initial data set of shed biofilm samples from three subjects with agent exposure on three separate days, the stored RNA samples were converted to cDNA and then sequenced all at once to avoid batch effects. This allowed the use of DESeq2 to determine differentially abundant 16S rRNAs.

Adonis2, which can partition distance matrices over sources of variation, was used to determine the contribution of sample donor and treatment exposure of the samples to the derived beta diversity as Bray Curtis Distance, among all samples, from the first three subjects. Unlike Adonis, order of variable entry for testing does not affect results.

Statistical analysis was performed using MaAsLin2 software for multivariable analysis of the sample data. The linear model method (LM) was used for the data analysis, with Total Sum Scaling normalization, log transformation, minimum abundance set to 4, minimum prevalence at 20%, and the FDR (q-value) less than 0.1. Categorical variables were summarized using frequencies and percentages. Continuous variables were presented as mean, and standard deviation.

To assess the differences in RNA and DNA levels, t-test was conducted when data followed a normal distribution. For bulk RNA and DNA level comparisons the Mann Whitney test was used due to non-normal distribution of some data.

### Data Availability

Raw NGS sequences, as FASTQ files, are available at the Sequence Read Archive of the National Center for Biotechnology Information, accession number PRJNA1076891.

What is claimed is:

1. A method of testing agents for activity to modulate oral microbes, the method comprising:
(a) providing a sample from a subject comprising shed oral biofilm;
(b) incubating a first portion of the sample with an agent, and a second portion of the sample without the agent, under a condition to yield a first incubated sample and a second incubated sample, and preserving a baseline portion of the sample prior to incubation; and
(c) determining level of at least one biological marker in the first incubated sample, the second incubated sample, and the baseline portion.

2. The method of claim 1, wherein the sample is collected in the form of saliva.

3. The method of claim 1, wherein the incubation of step (b) is conducted without subculturing the sample.

4. The method of claim 1, further comprising washing the sample prior to the incubation.

5. The method of claim 1, wherein the incubation is conducted under anaerobic conditions.

6. The method of claim 1, wherein the incubation is conducted for 4 hours or less.

7. The method of claim 1, wherein the incubation is conducted for 1 hour or less.

8. The method of claim 1, wherein the incubation is conducted in a rich medium.

9. The method of claim 1, wherein the biological marker comprises a nucleic acid, a protein, a metabolite, or a combination thereof.

10. The method of claim 9, wherein the nucleic acid marker comprises ribosomal RNA (rRNA), messenger RNA (mRNA), ribosomal DNA (rDNA), another DNA-based marker, or a combination thereof.

11. The method of claim 10, wherein the nucleic acid marker comprises full-length bacterial 16S rRNA or a region thereof.

12. The method of claim 10, wherein the nucleic acid marker comprises one or more bacterial mRNA indicative of gene expression changes in response to the agent.

13. The method of claim 10, wherein the nucleic acid marker comprises full-length bacterial 16S rDNA or a region thereof.

14. The method of claim 10, wherein determining the level of the nucleic acid marker comprises isolating total RNA or total DNA from the incubated sample.

15. The method of claim 10, wherein determining the level of the nucleic acid marker comprises performing one or more of: reverse-transcription PCR, quantitative PCR, digital PCR, next-generation sequencing, and RNA sequencing.

16. The method of claim 1, wherein a change in the level of the biological marker in the first incubated sample relative to the second incubated sample or to the baseline portion indicates that the agent modulates one or more oral microbes, including inhibiting, reducing, or increasing one or more oral microbial taxa.


SEQUENCE LISTING (2 sequences)

SEQ ID NO: 1 - DNA, 43 bp, synthetic construct
  source 1..43: mol_type = other DNA
acactgacgacatggttctacagtgtgycagcmgccgcggtaa

SEQ ID NO: 2 - DNA, 44 bp, synthetic construct
  source 1..44: mol_type = other DNA
tacggtagcagagacttggtctccggactacnvgggtwtctaat